(19) United States
(12) Patent Application Publication
(10) Pub. No.: US 2010/0105876 A1
(43) Pub. Date: Apr. 29, 2010

(54) Method for the oligomerisation of peptides

(76) Inventors: Alexey Terskikh, Lausanne (CH); Reto Crameri, Davos Platz (CH); Jean-Pierre Mach, Lausanne (CH); Jean-Marc Le Doussal, Paris Cedex (FR); Andrey Kajava, Lausanne (CH)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 10/132,533

(22) Filed: Apr. 25, 2002

Related U.S. Application Data
(63) Continuation of application No. 09/284,615, filed on Jun. 25, 1999, now abandoned.

(30) Foreign Application Priority Data
Oct. 28, 1996 (DE) 96810719.3

(51) Int. Cl. C07K 16/00; C12N 15/63; C12N 1/21

(52) U.S. Cl. 530/391.1; 435/320.1; 435/252.33

(57) ABSTRACT

The present invention concerns an oligomer comprising more than 2 units, wherein each unit comprises a peptidic domain capable of oligomerizing and a domain capable of binding to an acceptor (ligand), wherein the oligomerizing domain is not an antibody or a functional antibody fragment from the constant region. Also described is the use and synthesis of this oligomer.

Great strides made over the past years in understanding of molecular interactions in the realm of biomolecules such as proteins and nucleic acids benefited from the isolation of artificial polypeptide “ligands” with de novo binding activities to “receptors”. A powerful means of evolving artificial ligands is offered by the use of large polypeptide libraries, displayed on the surface of filamentous bacteriophage as a fusion with the phage coat proteins. In particular, isolation of new peptide ligands allowed, for example, to map antibodies binding sites, to find an important residues in HLA-DR molecules, to identify proteases substrates and inhibitors. Obviously, isolation of new peptide ligands with high affinity towards their target “receptors” can help to answer important biological questions and is of great interest for pharmaceutical development. However, apart from some exceptions, only low affinity (micromolar range) ligands were isolated from peptide libraries. This can be readily explained by a high degree of conformational freedom and a few number of contact residues within a short peptide molecule.

In order to improve this situation, a new type of high avidity binding molecule is constructed by harnessing the effect of multivalent interaction. In the current examples a short peptide ligand is expressed via a semi-rigid hinge region as a fusion with a coiled coil domain from Cartilage Oligomeric Matrix Protein (COMP), resulting in a pentameric multivalent binding molecule. In the case of a Pentabody (Pab-S) as described here, a peptide ligand (S) specific for the mouse B-cell lymphoma (BCL₁) surface idiotype, is selected from a phage displayed library. Pab-S molecule specifically bind the BCL₁ cells surface idiotype with an apparent avidity of about 1 nM, which corresponds to a 2×10⁵ fold increase, compared to the affinity of the synthetic peptide S itself. Equilibrium binding studies strongly suggest that high avidity of Pab-S is a result of multivalent interaction of its “peptide heads” and surface BCL₁ immunoglobulin receptor. As demonstrated by gel filtration chromatography, SDS-PAGE analysis and circular dichroism, Pab-S is a stable homopentamer of about 85 kDa, with the five chains crosslinked by disulfide bridges. Pab-S can be reversibly denatured, reconstituting upon renaturation its pentameric structure and full binding activity, providing an easy way to bring different peptide specificity within the same heteropentamer, thus bispecific or multispecific pentabodies.

The inventive oligomers have various unique features.
Target molecule specificity of inventive oligomer can be provided by a short peptide ligand, representing a “minimal” binding domain, where the primary structural information (i.e. the amino acid sequence) is sufficient for recognition. This demonstrates for the first time, that a low affinity peptide ligand (though specific) can be used to create a high avidity binding molecule.
A hinge region which dictates the geometry and the dynamic features of multivalent interaction can be easily introduced.
The inventive oligomers can be produced without extensive post translational modifications and, hence, can be produced easily in microorganisms like E. coli.
The peptide ligands can be attached to the C- or N-terminus of the oligomerizing domain, thus providing various binding geometry, with maximal distance between two peptide ligands (for example some attached to the C-terminus and some to the N-terminus in one oligomer).
If coiled coil α-helical domains are used, the resulting oligomers are in most cases well soluble due to the intrinsic structure.
The COMP domain can be used generally for oligomerization of compounds bound thereto.
It is a general method for the oligomerization of peptides.

### DETAILED DESCRIPTION OF THE INVENTION

The present invention concerns an oligomer comprising 2 or more than 2 units, wherein each unit comprises a peptidic domain capable of oligomerizing and a domain capable of binding to an acceptor (ligand), wherein the oligomerizing domain is not an antibody or a functional antibody fragment from the constant region.

A functional antibody fragment is, for example, the F_{C} domain or the constant region of the light or heavy chain.

In a preferred embodiment of the invention the inventive oligomer comprises more than 2 preferably more than 4 units and most preferably the inventive oligomer consist of 5 units.

In another preferred embodiment the individual units oligomerize spontaneously. A peptidic domain capable of oligomerizing is, e.g. a peptide that is known for its tendency to oligomerize like a pentamerization domain of the Cartilage Oligomeric Matrix Protein (Slavin & Strober (1978) Nature 272, 624-626). Especially preferred units can be separated from the oligomer and re-oligomerized without loss of activity.

In a further preferred embodiment of the invention each of monomeric units has less that 600 amino acids.

The acceptor, to which the inventive oligomer binds, may be of various origin like, for example, any protein that specifically binds to a partner like a substrate, an inhibitor, an activator, an antibody, a receptor and the like. A preferred acceptor is, for example, an antibody or a receptor. Examples for such preferred antibodies or receptors are BCL₁ cells from a B-cell lymphoma surface idiotype or any other receptor expressed on cell surface.

The inventive oligomer may comprise units having the same or different specificities in order to bind to the same or to distinct acceptors; wherein the domain capable of binding to an acceptor can be attached to the C- and/or N-terminus of the oligomerizing domain.

Embraced by the scope of the present invention is also the individual unit capable of oligomerizing as described above, a method for the synthesis of this unit, the expression vector used for this synthesis, as well as the host comprising said expression vector. Preferred hosts are of microbiological origin like E. coli.

There are several ways to synthesize the inventive units.
Via genetic engineering: An expression vector is constructed comprising an expression cassette for the complete inventive unit, that is expressed in a suitable host. Suitable expression cassettes can be prepared by standard techniques in genetic engineering. Beside the information necessary for the synthesis of the desired unit, the expression cassette may additionally contain a signal sequence that forces the secretion of the protein produced. It is also possible to express the inventive unit as a fusion protein with phage coat protein, especially those from filamentous phages and phagemids
Chemically: Due to the relatively short length of the individual units, they can be synthesized chemically, e.g. via solid phase synthesis.
Mixed: A part of the inventive unit is expressed by a suitable host and is connected with a chemically synthesized part. For example, the oligomerizing part is synthesized by a microorganism and is elongated by chemical synthesis to add a domain capable of binding to an acceptor.

The peptidic domain capable of oligomerizing and the domain capable of binding to an acceptor may be connected directly or via a spacer (hinge region) to provide, for example, a greater flexibility of the binding domain. For example, a proline-rich sequence of a spacer is supposed to prevent formation of secondary structure elements and as a consequence a fixed 3-D structure. Moreover, a spacer region like that is generally supposed to be quite rigid due to the conformational constrains of the proline residues, bringing a beneficial effect for the cooperativity of the multivalent binding. Accordingly, in another preferred embodiment of the invention, the peptidic domain capable of oligomerizing and the domain capable of binding to an acceptor are connected via a spacer, that comprises preferably a proline-rich region.

Furthermore, the C-terminus of some or all units of the oligomer may be modified by the addition of a further functional domain, like a marker, an enzymatic or a cytotoxic domain, or a domain that adds additional binding properties, for example to metal atoms or other structural compounds, that can be used, e.g., in affinity chromatography.

The individual units may be connected via disulfide bonds, e.g. spontaneously, or via one or more linker molecules. Such linker molecules are molecules bearing two or more reactive groups like —SH, —N₃, —COOH, —COBr, —COCl, —NH₂, —CHO, —CO—O—CO—, —CO—NH—CO—. Examples are N-5-azido-2-nitrobenzoyloxysuccinimide, p-azidophenazylbromide, p-azidophenyl glyoxal, N-4-(azidophenylthio)phthalimide, bis(sulfosuccinimidyl)suberate, bis-maleimidohexane, bis[2-(succinimidooxycarbonyloxy)ethyl]sulfone, 1,5-difluoro-2,4-dinitrobenzene, 4,4′-diisothiocyano-2,2′-disulfonic acid stilbene, dimethyl adipimidate, dimethyl pimelimidate, dimethyl suberimidate, dithiobis(succinimidylpropionate), disuccinimidyl suberate, disuccinimidyl tatrate, dimethyl 3,3′-dithiobispropionimidate, 4,4′-dithiobisphenylazide, 3,3′-dithiobis(succinimidylpropionate), ethyl-4-azidophenyl-1,4-dithiobutyrimidate, 1-azido-4-fluoro-3-nitrobenzene, N-hydroxysuccinimidyl-4-azidobenzoate, methyl-4-azidobenzoimidate, m-maleimidobenzoyl-N-hydroxysulfo-succinimide ester, N-hydroxysuccinimidyl-4-azidosalicylic acid, p-nitrophenyl-2-diazo-3,3,3-trifluoro propionate, N-succinimidyl(4-azidophenyl)-1,3′-dithiopropionate, sulfosuccinimidyl 2-(m-azido-o-nitrobenzamido)-ethyl-1,3′-dithiopropionate, N-succinimidyl-6(4′-azido-2′-nitrophenyl-amino)hexanoate, sulfosuccinimidyl 2-(p-azidosalicylamido)ethyl-1,3′-dithiopropionate, N-succinimidyl(4-iodoacetyl)aminobenzoate, succinimidyl 4-(N-maleimidomethyl)-cyclohexane-1-carboxylate, succinimidyl 4-(p-meleimidophenyl)-butyrate, N-succinimidyl 3-(2-pyridyldithio)propionate, bis[2-(sulfosuccinimidooxy-carbonyloxy)ethyl]sulfone, disulfosuccinimidyl tatrate, ethylene glycolbis(sulfosuccinimidylsuccinate), m-maleimidobenzoyl-N-hydroxysulfosuccinate), sulfosuccinimidyl (4-azidophenyldithio)-propionate, sulfosuccinimidyl 6-(4′azido-2′-nitrophenylamino)hexanoate, sulfosuccinimidyl (4-iodoacetyl)aminobenzoate, sulfosuccinimidyl 4-(N-maleimidomethyl)cyclohexane-1-carboxylate, sulfosuccinimidyl 4-(p-maleimidophenyl)butyrate and 2-iminothiolane.

Preferably, oligomerizing units capable of self-assembling are used. The linker molecules mentioned above may be also used additionally to stabilize the oligomer.

As shown in the example, spontaneous pentamerization of COMP assembly domain does not depend on disulfide bond formation, therefore, a preferred embodiment of the inventive oligomer represents a self-assembling molecule able to oligomerize in vivo and/or in vitro. Thus oligomerization of short peptides bypass folding problems and overcomes expression difficulties previously experienced during oligomerization of relatively complex proteins such as single chain Fv fragments. Moreover, a self-assembling of an oligomer allows it to undergo a reversible denaturation. Thus, heterooligomers can be readily obtained by mixing the inventive units with different specificity (i.e. with different peptide ligands) under denaturing conditions followed, e.g., by dialysis against any physiological buffer. This intrinsic property of the inventive oligomers opens an easy way to produce a chelating oligomer where two or more peptides directed against different epitopes on the same target molecule would be assembled together into a heteropentameric oligomer, allowing to benefit from the power of chelating effect, as it is recently demonstrated for the single chain Fv fragments. The same procedure can be used to produce heterooligomers with affinity to two or more receptors.

Binding studies of ¹²⁵I labeled Pab-S on BCL1 cells, for example, reveal an avidity of about 1 nM for the surface idiotype which is 2×10⁵ fold higher compared to IC₅₀ of the peptide S itself (200 μM). Pab-S can be reversible denatured (4M, urea 95° C., 15 min), regaining full binding activity after renaturation by dialysis in PBS. Data show that Pab-S has remarkable thermostability, retaining the binding activity even after autoclavation (120° C., 20 min).

Furthermore, it is expected that an oligomer harboring a peptide ligand with a nanomolar intrinsic affinity may attain a femtomolar range of avidity, approaching that of streptavidin-biotin interaction

Based on the properties of the inventive compounds, there are several applications for the inventive oligomers, that are all part of the invention.
Based on the equilibrium binding results of the example Pab-S it is evident that inventive oligomers are excellent molecules for eukaryotic cell, bacteria or viruses targeting; and especially for cell targeting. As it is shown for Pab-S, the avidity for the cell-surface immobilized receptor is much greater than the avidity for the soluble form of the same receptor. This property allows, for example, efficient targeting of the B-cell lymphomas, even in the presence of relatively high level of circulating idiotype antibody.
Based on the major feature of high avidity binding, especially for the surface immobilized antigens, the inventive oligomers can also be used as an inhibitor of protein-protein interactions, especially those occurring on the cell surface and in solution.
Heteropentamers can be produced by reassociating two or more different units of the inventive oligomers together. These heterodimers can bind to different epitopes on the same or different molecules and, e.g., can be used as a chelating and/or crosslinking agent.
The C-terminus of the molecules can be modified easily, e.g. by introducing peptide ligands with second specificity, a cytotoxic tail, a marker to identify acceptors, or a His-tail to chelate different toxic drugs (e.g. heavy metals) in order to deliver them to the target cells where they can be internalized, via an internalized surface receptor (e.g. immunoglobulin receptor) and kill the target cells.
It is possible, for example, to isolated a panel of C1q specific peptide ligands which can be used to produce a bispecific form of the inventive oligomers capable of C1q fixation and complement activation or complement inhibition.
Fusion of the peptide ligands to an Fc receptor results in a Nt-Ct bispecific form of the inventive oligomers capable of imitating the function of an Fc region from immunoglobulin.
Frameworks of the inventive oligomers can also be used to create random peptide libraries displayed on the filamentous bacteriophages. This allows rapid selection of the phage displayed inventive oligomers which then can be produced in a soluble form, in a suitable host.
Due to the relatively short length of the monomer polypeptide chain, the inventive oligomers can be synthesized chemically (e.g. standard Fmoc peptide chemistry, synthesis starts from the C-terminus). The N-terminal position of the peptide ligands allows to synthesized first the core molecule (e.g. the pentamerization domain and a linker) and then split the sample and continue synthesis with different peptide sequences on the N-terminus.
The same principle of chemical synthesis can be used to produce synthetic peptide library displayed on the inventive oligomers. Namely, synthesizing the N-terminal peptides at random (e.g. by Fmoc chemistry as described above), one can obtain a library of the peptides displayed on the inventive oligomer framework. This library can be absorbed on the target molecules or cells, eluted, purified (e.g. using 6×His tag) and the N-terminal peptide can be sequenced.
Furthermore, a framework of the inventive oligomers can be used to oligomerize peptides representing known B-cell epitope in order to obtain an efficient vaccines. Addition of different peptides known to enhance the immune response (such as T-helper epitopes or CR binding peptides derived from C3d) is also possible.
The inventive units can be expressed directly in a genetically engineered multicellular organism, in order to provide an in vivo production of the inventive oligomers.
The inventive units can be expressed in vitro using established transcription-translation systems.
The inventive units may be used in vitro as one of the binding reagents in enzyme immunoassays.
The inventive units can be used to induce apoptosis.
Due to the solubility and the high specificity of the compounds they can be used in gene therapy, e.g., to target certain receptors for activation or deactivation; or, if connected to toxic compounds, for their destruction.
Genes encoding the inventive units may be intracellularly expressed in order to inhibit the binding of transcription factors or gene regulating molecules as well as inhibit enzymatic activities.
The inventive units may be used to inhibit enzymatic activity or adhesion in order to prevent tumor metastization.
Furthermore, the epitopes of the inventive units can be used for vaccine development.
Due their high tendency for oligomerization the Cartilage Oligomeric Matrix Protein can be used generally for the pentamerization of low molecular weight compounds or peptides that are not part of the Cartilage Oligomeric Matrix Protein.

### Examples

Peptide synthesis. Peptides are synthesized using standard Fmoc solid phase chemistry on an Applied Biosystems 431A peptide synthesizer, lyophilized, re-dissolved in 50% acetic acid, purified by gel filtration through a G-25 sephadex column and analyzed by mass spectroscopy. For competition studies peptides are dissolved in PBS, adjusted to neutral pH, and peptide concentrations are determined by the method of Waddell based on an absorption difference at 215 and 225 nm.

Bacterial strains. E. coli TG1 (Wertman et al., (1986) Gene 49, 253-262) is used for propagation of plasmids and phages and E. coli SG13009 (QIAEX) is used for production of fusion proteins.

Labeling. Typically, Pab-S (17 μg, 0.2 nmol), B1 IgG (75 μg, 0.5 nmol) or B1 Fab′ (50 μg, 1 nmol) are labeled in PBS with 100 μCi (1 μCi=37 MBq) of ¹²⁵I in a in Iodo-Gen (Biorad, 10 μg) coated tubes for 20 min (2 h for Pab-S) at 4° C. Uncoupled iodine is removed by gel filtration on a PD-10 column (Pharmacia). About 40% of the radioactivity for Pab-S and 70% for B1 IgG and B1 Fab′ is recovered. Specific activity ranges from 70 to 200 μCi/nmol.

RIA competition on plastic. Polyvinil 96-well plates are coated with BCL1 IgM at 3 μg/ml in PBS for 16 h at 4° C. and blocked with 2% MPBS for 1 h at 37° C. Different amounts of peptides are incubated with 10 nCi of ¹²⁵I labeled B1 IgG for 1 h at 37° C. The wells are washed with PBS containing 0.1% of Tween-20 and the amounts of radioactivity retained is measured with an automated multichannel Cobra II γ-counter.

### Example 1

### Selection of Peptides Specific for BCL1 Surface Immunoglobulin Receptor

Cells and antibodies. The BALB/c derived B cell lymphoma BCL₁ (Efimov et al., (1994) FEBS Letters 341, 54-58), and the mouse hybridoma B1, secreting an anti-idiotype monoclonal IgG₁ antibody (B1 IgG), are kindly provided by Dr. Kris Thielemans (Medical School, VUB, Brussels, Belgium). BCL₁ cells are propagated in BALB/c mice using i.p. injection of 10⁶ cells. The BCL₁ soluble IgM is purified from the serum of a splenomegalic mouse by precipitation with 50% ammonium sulfate, followed by anion exchange and gel filtration chromatography (Mono Q and Superdex 200, Pharmacia). The B1 IgG is purified by protein G-Sepharose (Pharmacia). Fab′ fragments are obtained by limited digestion with pepsin, followed by reduction and alkylation, using standard methods.

Peptide selection. Peptide ligands specific for mouse B-cell lymphoma (BCL₁) idiotype, selected from three different peptide libraries displayed on two filamentous bacteriophages libraries of about 10⁷ independent members displaying random hexapeptides called Smith (Scott & Smith (1990) Science, 249, 386-390), and Doorbar (Doorbar & Winter (1994) J. Mol. Biol., 244, 361-369), are used. In addition a combinatorial library of about 10¹² independent members displaying a tandem of random decapeptides, called Fisch (Fisch et al., Proc. Natl. Acad. Sci. USA (1996), 93, 7761-7766), is used.

Screening of the phage display libraries is performed essentially as described by Fisch et al., (Proc. Natl. Acad. Sci. USA (1996), 93, 7761-7766). Briefly, the purified BCL₁ IgM is coated on immunotubes (Nunc) at 50 μg/ml in PBS (50 mM phosphate, 150 mM NaCl, pH 7.4). About 10¹² colony forming units (c.f.u.) of phage from each library are used for each round of selection. After three rounds of panning, individual clones are isolated, and binding of phage to BCL₁ IgM coated on plastic (3 μg/ml) is measured by ELISA (Barrett et al., (1992) Anal. Biochem. 204, 357-364) using horseradish peroxidase conjugated anti-M13 antibody (Pharmacia). Plates coated with mouse IgG or with human IgM, are used as negative controls. Specific inhibition of phage binding to BCL₁ IgM, is performed by addition of B1 IgG at 100 μg/ml. The DNA fragments encoding the selected peptides are amplified by PCR and sequenced as described Fisch et al., (Proc. Natl. Acad. Sci. USA (1996), 93, 7761-7766).

Specific phages are selected on the purified BCL₁ IgM, individual phage clones are isolated, and the DNA fragments encoding peptides are amplified by PCR and sequenced (Table 1). Interestingly, two distal Cys residues, potentially capable of forming a disulfide bonded loop, are found in the hexapeptide selected from the Smith library. Idiotype specificity of selected peptides is demonstrated by inhibition of phage binding with the anti-idiotype antibody, B1 IgG.

TABLE 1
Amino acid sequences of selected idiotype specific
peptide ligands for BCL1 immunoglobulin receptor.

| | | Sequ | |
|---|---|---|---|
| Library/sequence* | Selected peptides^{†} | ID | name |
| Doorbar/ | [symbol] PYDKYE^{‡} | 1 | D |
| X6-PNDKYE-g3p | | | |
| Smith/ | ADGA [symbol] | 2 | S |
| ADGA-X6-g3p | | | |
| Fisch/ | [symbol] [symbol] ALLRYTCPT^{¶} | 3 | F |
| X10-ALLRY-X10-g3p | | | |

*Names and amino acid sequences of the phage displayed peptide libraries; X6 and X10 denotes random hexa- and deca-peptide correspondingly.
^{†}Peptides selected from random libraries are in bold type, framework residues are in normal type, corresponding synthetic peptides are in italic type.
^{‡}An unusual N to Y substitution in the constant part of the peptide from Doorbar library is observed.
^{¶}First ten amino acid of the peptide selected from Fisch library was synthesized.

Synthetic peptides corresponding to the selected sequences are prepared as outlined in Table 1. Cysteins containing peptide S is completely oxidized on air loosing two hydrogen atoms, as shown by Mass Spectroscopy, which is consistent with the cyclization of the peptide. All synthetic peptides are tested for binding inhibition of ¹²⁵I labeled B1 IgG to BCL₁ IgM coated on plastic. The D and the S peptides inhibit binding with an IC₅₀ of about 60 μM and 200 μM, respectively. No inhibition is seen with peptide F up to 2 mM, which is used, thereafter, as a negative control. Interestingly, substitution of Cys residues by Ser in peptide S results in a 10 fold loss of affinity, suggesting that disulfide bounded turn conformation of the peptide is favorable for binding to BCL₁ IgM idiotype.

### Example 2

### Pentabody. Molecular Design and Gene Construct

Pentabody fusion protein, is designed consisting of four distinct parts.
First, a selected peptide specific for BCL₁ IgM idiotype represented the N-terminal binding domain.
Second, a 24 amino acid sequence selected from a long camel Ig hinge region (Hamers-Casterman et al., (1993) Nature 363, 446-448) is placed to provide a space necessary for multivalent binding.
Third, the hinge region is followed by a 55 amino acid long pentamerization domain, a modification of the described coil-coiled COMP assembly domain (Slavin & Strober (1978) Nature 272, 624-626), known to spontaneously form a five-stranded α-helical bundle. Two cystein residues present at the C-terminal of the wild type sequence allowed the formation of inter-chain disulfide bridges. Based on the recently described model of COMP assembly domain (Kajava (1996) Proteins 24, 218-226) two substitutions Lys29Cys and Ala30Cys are introduce in the modified version, making a possible the formation of additional disulfide bonds near the N-terminal part of the assembly domain in order to further stabilize the α-helical bundle.
Forth, a sequence encoding six histidine residues is placed at the C-terminus of the fusion molecule to facilitate the protein purification via the metal-chelating affinity chromatography.

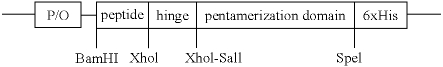

The chimeric genes coding for the different Pab are constructed as designed (see below) and named Pab-S, Pab-D and Pab-F, where the last letters stay for the S, D, and F peptide, respectively (see table 1).

Essentially, the DNA sequences encoding the BCL₁ idiotype specific peptides, as well as the hinge region, are assembled from oligonucleotide duplexes. The COMP pentamerization domain is amplified by PCR simultaneously introducing point mutations, and the fusion genes are assembled into a modified pDS-78 expression vector.

Plasmid construction. The construction of plasmid p3bCOMP encoding a 64 amino acid COMP assembly domain is described in (Slavin & Strober (1978) Nature 272, 624-626). All further constructs are made using standard methods of DNA manipulation (Sambrook et al., (1990) Molecular Cloning: A Laboratory Manual (Cold Spring Harbor Lab. Press, Painview, N.Y.)). The DNA fragment encoding the COMP domain (residues 26-80, amino acids numbering as in Slavin & Strober (1978) Nature 272, 624-626) is amplified by PCR from p3bCOMP template using COMP-Xho-BACK (5′-AG ATC CTC GAG GGT GGA GAC TGC TGC CCA CAG ATG CTT AGA; SEQ ID NO 4) and COMP-Spe-FOR (5′GC ACT AGT AGA ACC GCC ACC CGG GGT; SEQ ID NO 5) primers. Thus the resulting product contains two amino acid substitutions Lys29Cys and Ala30Cys as well as XhoI and SpeI sites at the 5′ and 3′ end, respectively. The DNA duplexes encoding the peptides S, F, and D, are prepared by annealing the oligonucleotides S-up (5′GA TCC GCT GAC GGC GCT TGC CGT ACC CCG TGG TGC GGT C; SEQ ID NO 6) with S-low (5′TC GAG ACC GCA CCA CGG GTT ACG GCA AGC GCC GTC AGC G; SEQ ID NO 7), F-up (5′GA TCC ACT GCT GCA GGT CTG TGC GAA TCC GAC CAG C; SEQ ID NO 8) with F-low (5′TC GAG CTG GTC GAA TTC GCA CAG ACC TGC AGC AGT G; SEQ ID NO 9) and D-up (5′GA TCC TCT GTT TGG CGT TGG CTG CCG TAC GAC AAA TAC GAA C; SEQ ID NO 10) with D-low (5′TC GAG TTC GTA TTT GTC GTA CGG CAG CCA ACG CCA AAC AGA G; SEQ ID NO 11). All three duplexes contain BamHI and XhoI cohesive ends on 5′ and 3′ end, respectively. By means of three-part ligation, the duplexes encoding peptides and the PCR amplified COMP domain, restricted with XhoI and SpeI enzymes, are joint together in the pDS78 vector, linearized with BamHI and SpeI enzymes, in front of a 6 histidine tail present in the vector (Stuber et al., in Immunological Methods, eds. Lefkovits, I. & Perris, B. (Academic Press, New York), pp. 121-152). This generated pSC6H pDC6H and pFC6H plasmids which encode S, D and F peptides respectively. A DNA fragment encoding the 24 amino acid hinge region [(PQ)₂PK(PQ)₄PKPQPK(PE)₂], called PX, is prepared by annealing of PX-up (5′TC GAG CGG CAG CCG CAG CCG AAA CCG CAG CCG CAG CCG CAG CCG CAG CCG AAA CCG CAG CCG AAA CCG GAA CCG GAA G; SEQ ID NO 12) and PX-low (5′TC GAC TTC CGG TTC CGG TTT CGG CTG CGG TTT CGG CTG CGG CTG CGG CTG CGG CTG CGG TTT CGG CTG CGG CTG CGG C; SEQ ID NO 13) oligonucleotides encoding the plus and minus strands of the duplex and contain XhoI and SalI cohesive ends on 5′ and 3′ end, respectively. The PX duplex is ligated into XhoI linearized, dephosphorylated pSC6H pDC6H and pFC6H plasmids, to generate the expression vectors pSPXC6H (DSM 11236), pDPXC6H and pFPXC6H, respectively. The final constructs are verified by dideoxynucleotide sequencing using Sequenase 2.0 (United States Biochemical).

### Example 3

### Expression and Purification of Pentabodies

Pab fusion proteins are expressed in E. coli SG13009. The cultures are grown on a shaker at 37° C. up to OD₆₀₀˜0.5, then 1 mM IPTG is added to induce protein synthesis, followed by further 4 h incubation at 30° C. Bacteria are collected by centrifugation (8000×g, 15 min, 4° C.) and frozen at −70° C. Bacterial pellet is resuspended in PBS pH 7.4, 1 mM EDTA, 1 mg/ml lysozyme, incubated for 30 min at room temperature and subjected to three rounds of freezing/thawing (liquid nitrogen/37° C.). The lysate is incubated for 15 min at RT with 0.1 mg/ml of DNAse I and after centrifugation (23,000×g, 15 min, 4° C.) the supernatant is collected. Imidazol is added to a final concentration of 5 mM and the recombinant protein is absorbed on 2 ml of Ni-NTA resin (QIAGEN), equilibrated in 5 mM imidazol, PBS pH 7.4. After extensive wash with PBS containing 5 mM and 20 mM imidazol, retained proteins are eluted with PBS containing 250 mM imidazol. After extensive dialysis against PBS, 1 mM EDTA, proteins are concentrated 5 times with Centriprep 10 concentrator (Amicon), aliquoted and stored at −20° C.

The Pab-S and Pab-F fusion proteins are expressed at high levels (>30 mg/l), allowing efficient one step metal-chelating affinity chromatography under native conditions. The amount of soluble Pab-D protein is lower than that of Pab-S and Pab-F.

Affinity purified Pab-S and Pab-F molecules are fractionated by FPLC gel filtration. A single elution peak corresponding to a protein of about 85 kDa is observed for Pab-S, whereas two major elution peaks corresponding to the proteins of about 90 and 180 kDa are observed for Pab-F. The high molecular weight peak presumably resulted from dimer formation via unsaturated cystein present in peptide F. In both cases, fractions of the elution peak corresponding to a 85-90 kDa protein are collected, pooled and used for binding studies.

### Example 4

### Equilibrium Binding Studies

Cell dilution experiments and homogenous competition curves (i.e. competition for the cell binding between the labeled and the unlabeled forms of the same ligand) are described by a monovalent equilibrium model. The free (F) and bound (B) radioactivity is corrected from the non-immunoreactive fraction (NIF) and non-specific binding (NSB) respectively. Both are expressed in mol/l, taking into account the specific activity of each isotopic dilution (B and F, respectively). The parameters Kd, R, NSB and NIF, where R is the molarity of binding sites and Kd is the dissociation equilibrium constant, are fitted by non-linear regression of the corrected Scatchard equation

$$\frac{B - N S B}{F - N I F} = \frac{R}{Kd} - \frac{( B - N S B )}{Kd}$$

to the experimental data.

To determine maximal immunoreactivity, ¹²⁵I-labeled Pab-S, B1 IgG or B1 Fab′ (20 nCi) are incubated with serial dilutions of freshly harvested BCL1 cells (0.3 to 100×10⁵ cells per 100 μl). For competition assays, the labeled compounds are incubated with serial dilution of unlabeled competitors and BCL1 cells (25, 50 or 100×10⁵ per 100 μl). Comparative experiments are performed the same day with the same batch of cells, in 96-wells plates, in triplicates, in a final volume of 150 μl of PBS supplemented with 1 mg/ml of Bovine Serum Albumin (BSA), at 4° C., under agitation and for 2.5 h. After centrifugation, the amounts of both free and bound ¹²⁵I are measured as above from an aliquot of the supernatant and of the pellet washed once with PBS (4° C.).

In preliminary solid phase competition assay Pab-S is found to compete the binding of ¹²⁵I labeled B1 IgG to BCL₁ IgM idiotype coated on plastic. However, since spatial arrangement of target molecule on the surface may influence multivalent binding, the equilibrium binding parameters of Pab-S are determined directly on live BCL₁ cells, which represent a more relevant biological surface. Purified Pab-S is labeled with ¹²⁵I and is shown to bind to BCL1 idiotype on the cell surface. It can be competed by unlabeled Pab-S, B1 IgG, and by a much higher concentration of peptide S, but not by the control Pab-F pentamer. The competitions of ¹²⁵I-labeled Pab-S binding by unlabeled Pab-S at 3 different cell concentration and a cell dilution experiment are used together for curve fitting to obtain the equilibrium binding parameters.

The apparent equilibrium binding constant of Pab-S is found to be similar to that of B1 IgG (around 1 nM) which represents a 2×10⁵ fold increase in avidity compared to the peptide S itself (IC₅₀ around 200 μM). As control, the same binding experiments are performed with ¹²⁵I labeled B1 IgG and B1 Fab′ fragment. The obtained constants are consistent with already published data. The Scatchard equilibrium binding constants are:

| Fragment | Kd [nM] |
|---|---|
| Pab-S | 1.0 ± 0.4 |
| IgG | 0.8 ± 0.4 |
| Fab′ | 3.5 ± 0.1 |

The lower molarity of binding sites found for Pab-S compared to B1 IgG and Fab provides evidence for a multivalent nature of Pab-S binding.

In another series of experiments we compare the capacity of soluble BCL₁ IgM to competed the binding of either Pab-S or B1 IgG to BCL₁ cells. The results showed that much higher concentration of soluble BCL₁ IgM is needed to compete for Pab-S as compared to B1 IgG (100 vs 4 nM for 80% displacement). These data indicate a higher avidity of Pab-S for the cell surface-immobilized BCL₁ IgM, compared to the soluble BCL₁ IgM. Importantly, no inhibition of Pab-S binding to BCL₁ IgM idiotype is observed in the presence of up to 30% (v/v) of human or mouse BALB/c serum.

### Example 5

### Biochemical Characterization of Pab-S Chimeric Protein

The concentration of purified Pab-S is determined by the method of Waddell, based on an absorption difference at 215 and 225 nm as well as by Bradford protein assay (Bio-Rad). Pab-S molecule is characterized using FPLC gel filtration, SDS-PAGE and CD spectrometry. Oxidized form of Pab-S with inter-chain disulfide bonds is formed by air oxidation during the purification procedure and dialysis against PBS. Completely reduced form is obtained by incubation with 100 mM DTT at 37° C. for 30 min, followed by extensive dialysis against PBS, 1 mM EDTA, 1 mM β-mercaptoethanol.

Under non-denaturing conditions, a single elution peak, corresponding to a protein of about 85 kDa is detected for both oxidized and reduced forms of Pab-S after gel filtration on Superdex G200 column (equilibrated in PBS, 1 mM EDTA, ±1 mM β-mercaptoethanol, elution is monitored at 280 nm with an integrated UV detector (the proteins are analyzed on 10-15% gradient). Under denaturing conditions (Pab-S is denatured by boiling for 20 min in 4 M urea and 100 mM DTT and renatured by extensive dialysis against PBS, 1 mM EDTA, 1 mM β-mercaptoethanol) in SDS-PAGE, a major protein with apparent molecular weight of about 85 kDa is observed for the oxidized form of Pab-S molecule, whereas a protein with apparent molecular weight of about 17 kDa is observed for the reduced form of Pab-S, in agreement with a covalent pentameric structure. A minor band with apparent mobility of about 68 kDa observed for the oxidized Pab-S corresponds to a partially reduced pentamer, where only four chain out of five are covalently link by disulfide bridges. From the analysis of the CD spectrum of Pab-S pentamer an α-helical content of about 54% is determined by fitting the experimental data with a set of reference proteins as described in Vogel (Biochemistry (1987), 26, 4562-72). This is in a good agreement with the length of the α-helical coiled-coil domain within the entire molecule. Taken together these data indicate that Pab-S molecule is a stable homopentamer of about 85 kDa with a monomer subunits of about 17 kDa held together by in α-helical coiled-coil bundle, where the five chains are covalently crosslinked by disulfide bonds.

It is known that coiled coil structures can undergo reversible denaturation. Indeed, the Pab-S molecule is denatured in urea at 95° C. under reducing conditions and refolded into the pentamer by simple dialysis against PBS, as demonstrated by FPLC gel filtration of renatured Pab-S labeled with ¹²⁵I. Noteworthy, renaturation of Pab-S restores full binding activity as shown by binding competition on BCL1 cells.

To visualize the spatial arrangement of Pab-S molecular modeling of its three-dimensional structure is undertaken. Pab-S molecule is modeled using the computer graphics program TOURBO-FRODO (Roussel & Cambillan (1989) in Silicon Graphics Geometry Partner Directory (Fall 1989), eds. Silicon Graphics (Silicon Graphics, Mountain View, Calif.), pp. 77-78). The structure is further refined by the X-PLOR program version 3.1 (Brunger. (1992) in X-PLOR version 3.1 A system for X-ray Crystallography and NMR, (New Haven, Yale University Press)) using the following procedure: a 5 ps molecular dynamics simulation at 300 K and then 1,000 steps of conjugate gradient minimization, carried out with 1/r dependent dielectric constant.

The stereochemical analysis shows that frequently occurring type I conformation of β-turn allows formation of disulfide bond between the two cysteins of peptide S. This conformation is chosen, as the most likely, for the peptide ligand. For the hinge region a molecular dynamic simulation with subsequent energy minimization resulted in a set of possible conformations. Out of it, a “good-looking” conformation is arbitrary chosen. Finally, previously modeled five-stranded α-helical coiled-coil structure is taken for the pentamerization domain. The analysis suggests a fivefold symmetrical structure of Pab-S protein. The analysis of the modeled Pentabody structure shows that such a molecule should be capable of simultaneous binding to five surface receptor, provided they are located up to 80 Å apart from each other. This criterion is largely satisfied in the case of the surface immunoglobulin receptor, whose variable domains can be spaced at about 50 Å, judging by their van der Waals contact radii.

### Example 6

### Use of Pentabody in in Vitro for Assays

To measure the level of an antigen of interest such as prostrate specific antigen (PSA) which is an enzyme of the kallikrein family, peptides displayed on a phage display library may be screened for those binding specifically the antigen of interest, such as PSA, with a great specificity and avidity. Screening may be as described in the examples above, particularly example 1. Pentabody expressing the selected peptide(s) in pentameric form may be constructed, expressed and purified as described in the examples above, particularly examples 2 and 3. The selected pentabodies may be labeled with a marker enzyme such as peroxidase.

Antibodies specific for the antigen of interest, such as PSA, may be immobilized on a solid phase such as plates or polystyrene balls. The immobilized, unsolubized antibodies may be incubated with the test fluid, such as serum or other biological fluid for the first step of immunoabsorption. After incubation, the solid phase may be washed.

The selected pentabody labeled with a marker enzyme may be incubated with the immunoabsorbed antigen, such as PSA. Then the substrate for the marker enzyme conjugated to the pentabody and a chromogen may be added. The optical density may then be measured. The optical density of the chromogen will be proportional to the amount of antigen, such as PSA, present. The assays of the invention may also comprise radioimmunassays using the pentabodies of the invention, the above approach and methods known in the art.

Deposition of Microorganisms

The following microorganisms are deposited according to the Budapest Treaty with the DSM—Deutsche Sammlung von Mikroorganismen and Zellkulturen GmbH, Mascheroder Weg 1b, D-38124 Braunschweig:

| | | | |
|---|---|---|---|
| E. coli SG13009 | pSXC6H | 21.10.1996 | DSM11236 |

1. An oligomer comprising 2 or more than 2 units, wherein each unit comprises a peptidic domain capable of oligomerizing and a domain capable of binding to an acceptor, wherein the oligomerizing domain is not an antibody or a functional antibody fragment from the constant region.

2. An oligomer according to claim 1 comprising more than 4 units.

3. An oligomer according to claim 1 consisting of 5 units.

4. An oligomer according to claim 1, wherein the acceptor is a antibody or a receptor.

5. An oligomer according to claim 1, wherein each of said units has less that 600 amino acids.

6. An oligomer according to claim 1, wherein the peptidic domain capable of oligomerizing and the domain capable of binding to an acceptor are connected via a spacer (hinge region).

7. An oligomer according to claim 6, wherein the spacer comprises a proline-rich region.

8. An oligomer according to claim 1, wherein at the C-terminus of some or all units a further functional domain is attached.

9. An oligomer according to claim 1, wherein the individual units oligomerize spontaneously.

10. Use of the pentamerization domain of the Cartilage Oligomeric Matrix Protein for the pentamerization of low molecular weight compounds or peptides that are not part of the Cartilage Oligomeric Matrix Protein.

11. An oligomer according to claim 1, wherein the oligomerizing domain is the pentamerization domain of the Cartilage Oligomeric Matrix Protein.

12. An oligomer according to claim 1 comprising units that bind to distinct acceptors.

13. A unit capable of oligomerizing according to claim 1.

14. Use of an oligomer according to claim 1 for identification and/or marking of acceptors.

15. Use of an oligomer according to claim 1 for eukaryotic cell, bacteria or viruses targeting.

16. Use of an oligomer according to claim 1 for cell targeting.

17. Use according to claim 16 for targeting of B-cell lymphomas.

18. Use of an oligomer according to claim 1 for the inhibition of protein-protein interactions.

19. Use of an oligomer according to claim 12 as a chelating agent.

20. Use of an oligomer according to claim 12 as a crosslinking agent.

21. Use of oligomers according to claim 1 for the construction of libraries.

22. Use of oligomers according to claim 1 for the induction of apoptosis.

23. Use of oligomers according to claim 1 for the intracellular inhibition of transcription factor binding, gene regulating molecules and/or enzymatic activities.

24. Use of oligomers according to claim 1 for prevention of tumor metastatization.

25. Use of oligomers according to claim 1 in vitro as one of the binding reagents in an enzyme immunoassay.

26. Use of oligomers according to claim 1 in vitro as one of the binding reagents in radioimmunoassays.

27. Method for the synthesis of a unit according to claim 13.

28. An expression vector for the synthesis of a unit according to claim 13.

29. A host, comprising an expression vector for the synthesis of a unit according to claim 13.

30. A microbiological host, comprising an expression vector for the synthesis of a unit according to claim 13.

31. A method for the production of an oligomer according to claim 1.

32. An oligomer according to claim 1, wherein the epitopes are used for vaccine development.


SEQUENCE LISTING (13 sequences)

SEQ ID NO: 1 - protein, 12 aa, Artificial Sequence
  Synthetic peptide ligand specific for mouse BCL1 idiotype selected from peptide libraries dispalyed on filamentous bacteriophage displaying random hexapeptides or decapeptides
SVWRWLPYDKYE

SEQ ID NO: 2 - protein, 10 aa, Artificial Sequence
  Synthetic peptide ligand specific for mouse BCL1 idiotype selected from peptide libraries dispalyed on filamentous bacteriophage displaying random hexapeptides or decapeptides
ADGACRNPWC

SEQ ID NO: 3 - protein, 19 aa, Artificial Sequence
  Synthetic peptide ligand specific for mouse BCL1 idiotype selected from peptide libraries dispalyed on filamentous bacteriophage displaying random hexapeptides or decapeptides
TAAGLCEFDQALLRYTCPT

SEQ ID NO: 4 - DNA, 41 bp, Mus musculus
  primer_bind (1)...(41): COMP-Xho-BACK primer
agatcctcgagggtggagactgctgcccacagatgcttaga

SEQ ID NO: 5 - DNA, 26 bp, Mus musculus
  primer_bind (1)...(26): COMP-Spe-FOR primer
gcactagtagaaccgccacccggggt

SEQ ID NO: 6 - DNA, 39 bp, Mus musculus
  primer_bind (1)...(39): S-up primer
gatccgctgacggcgcttgccgtaccccgtggtgcggtc

SEQ ID NO: 7 - DNA, 39 bp, Mus musculus
  primer_bind (1)...(39): S-low primer
tcgagaccgcaccacgggttacggcaagcgccgtcagcg

SEQ ID NO: 8 - DNA, 36 bp, Mus musculus
  primer_bind (1)...(36): F-up primer
gatccactgctgcaggtctgtgcgaatccgaccagc

SEQ ID NO: 9 - DNA, 36 bp, Mus musculus
  primer_bind (1)...(36): F-low primer
tcgagctggtcgaattcgcacagacctgcagcagtg

SEQ ID NO: 10 - DNA, 42 bp, Mus musculus
  primer_bind (1)...(42): D-up primer
gatcctctgtttggcgttggctgccgtacgacaaatacgaac

SEQ ID NO: 11 - DNA, 42 bp, Mus musculus
  primer_bind (1)...(42): D-low primer
tcgagttcgtatttgtcgtacggcagccaacgccaaacagag

SEQ ID NO: 12 - DNA, 78 bp, Mus musculus
  primer_bind (1)...(78): PX-up primer
tcgagcggcagccgcagccgaaaccgcagccgcagccgcagccgcagccgaaaccgcagccgaaaccggaaccggaag

SEQ ID NO: 13 - DNA, 78 bp, Mus musculus
  primer_bind (1)...(78): PX-low primer
tcgacttccggttccggtttcggctgcggtttcggctgcggctgcggctgcggctgcggtttcggctgcggctgcggc